(19) United States
(12) Patent Application Publication
(10) Pub. No.: US 2012/0295979 A1
(43) Pub. Date: Nov. 22, 2012

(54) USE OF SULINDAC FOR PROTECTING RETINAL PIGMENT EPITHELIAL CELLS AGAINST OXIDATIVE STRESS

(75) Inventors: Howard M. PRENTICE, Boca Raton, FL (US); Herbert Weissbach, Boynton Beach, FL (US); Janet Blanks, Boca Raton, FL (US)

(73) Assignee: Florida Atlantic University, Boca Raton, FL (US)

(21) Appl. No.: 13/463,440

(22) Filed: May 3, 2012

Related U.S. Application Data
(60) Provisional application No. 61/482,036, filed on May 3, 2011.

(51) Int. Cl. A61K 31/192; A61P 39/06; A61P 27/02

(52) U.S. Cl. 514/569

(57) ABSTRACT

Compositions and methods described herein are based on the use of the drug sulindac, a non steroidal anti-inflammatory drug (NSAID), for protecting retinal pigment epithelial cells against oxidative stress which is a major component of macular degeneration. Described herein is a new use for sulindac.

### CROSS-REFERENCE TO RELATED APPLICATIONS

This application claims the benefit of Provisional Application Ser. No. 61/482,036, filed May 3, 2011, which is hereby incorporated by reference in its entirety, for all purposes, herein.

### FIELD OF THE INVENTION

The invention relates generally to the fields of ophthalmology, molecular biology, and medicine.

### BACKGROUND

The retinal pigment epithelial (RPE) layer is one of the major areas affected by oxidative stress in ocular diseases, including macular degeneration. Therapeutic agents for treating such diseases are needed.

### SUMMARY

In the experiments described herein, the non-steroidal anti-inflammatory compound sulindac was tested for protection against oxidative stress induced damage in RPE cells. Besides its known anti-inflammatory activity, recent studies have shown that sulindac can protect cardiac cells against oxidative damage by a preconditioning mechanism. Compositions and methods described herein are based on the use of the drug sulindac, a non steroidal anti-inflammatory drug (NSAID), for protecting retinal pigment epithelial cells against oxidative stress which is a major component of macular degeneration. Described herein is a new use for sulindac.

Unless otherwise defined, all technical terms used herein have the same meaning as commonly understood by one of ordinary skill in the art to which this invention belongs.

The terms “patient,” “subject” and “individual” are used interchangeably herein, and mean a mammalian (e.g., human) subject to be treated and/or to obtain a biological sample from.

As used herein, the term “treatment” is defined as the application or administration of a therapeutic agent to a patient or subject, or application or administration of the therapeutic agent to an isolated tissue or cell line from a patient or subject, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a composition as described herein effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutically acceptable” component is one that is suitable for use with humans and/or animals without undue adverse side effects (such as toxicity, irritation, and allergic response) commensurate with a reasonable benefit/risk ratio.

The terms “dosing” and “treatment” as used herein refer to any process, action, application, therapy or the like, wherein a subject, particularly a human being, is rendered medical aid with the object of improving the subject's condition, either directly or indirectly.

The term “therapeutic compound” as used herein refers to a compound useful in the prophylaxis or treatment of oxidative damage or stress, e.g., macular degeneration initiated by oxidative damage or stress.

Accordingly, described herein is a method of protecting retinal cells from damage from oxidative stress in a subject (e.g., a human). The method includes administering to the subject a composition including sulindac in an amount effective to protect retinal cells from damage from oxidative stress. In some embodiments, the subject is a human with macular degeneration. The composition can be, for example, an eye drop formulation. The retinal cells can be, for example, retinal pigment epithelial (RPE) cells.

Also described herein is a pharmaceutical composition including sulindac in an amount effective to protect RPE cells from damage from oxidative stress and a pharmaceutically acceptable carrier, the composition formulated as an eye drop formulation.

Yet further described herein is a kit for treating macular degeneration in a subject. The kit includes: an eye drop formulation including sulindac in an amount effective to protect RPE cells from damage from oxidative stress and a pharmaceutically acceptable carrier; instructions for use; and packaging. Although compositions and methods similar or equivalent to those described herein can be used in the practice or testing of the present invention, suitable compositions and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is an illustration of the structure of sulindac and its analogues.

FIG. 2 is a graph showing a “kill curve” of ARPE-19 cells grown in 96 well plates.

FIG. 3 is a graph showing results from experiments in which treating ARPE 19 cells with sulindac prior to TBHP exposure prevented cell death caused by oxidative stress.

FIG. 4 is a graph showing that the loss of cell viability by H₂O₂ induced oxidative stress can be reduced by preincubating retina cells with sulindac.

FIG. 5 is a graph showing comparative cellular viability of ARPE-19 cells treated with sulindac, sulindac sulfone and untreated (control) cells.

FIG. 6 is a pair of images highlighting the difference in vision caused by damage to the macula in the eyes of patients suffering from AMD.

FIG. 7 is a graph showing that sulindac protects RPE cells against TBHP oxidation and UV treatment. A) RPE cells were preincubated with 125 uM and 200 uM concentrations of sulindac for 24 hours. B) RPE cells exposed to 1200 mj of UVB radiation.

FIG. 8 is a graph showing the protective effect of sulindac sulfone in protecting RPE cells against UVB radiation.

FIG. 9 is a pair of graphs showing the effect of PKC inhibitors on sulindac protective effect of sulindac on RPE cells exposed to 1200 mj of UVB radiation A) Reversal by the PKC broad spectrum inhibitor chelerythrine B) Effect of specific inhibitors of PKCε and PKCδ.

FIG. 10 is a pair of photographs of Western blots showing that two late phase markers of preconditioning, iNOS (FIG. 10A) and Hsp27 (FIG. 10B) were upregulated in ARPE19 cells treated with sulindac.

FIG. 11 is a graph showing that Sildenafil, a known IPC agent, can replace sulindac in protecting RPE cells against 1200 mj UVB damage.

FIG. 12 is a graph showing the effect of the PKG inhibitor on the sulindac protection of RPE cells.

### DETAILED DESCRIPTION

Damage to RPE cells by oxidative stress is a central factor in the initiation and progression of macular degeneration and protection of the RPE cells against oxidative stress is an important therapeutic strategy for preventing the disease. Described herein are uses of sulindac, a known NSAID, for protecting against RPE cell damage from oxidative stress. Sulindac has been shown to protect normal cells from oxidative stress and to selectively enhance the killing of cancer cells exposed to agents that affect mitochondrial function leading to oxidative stress. In normal heart cells sulindac protected cardiac myocytes from oxidative damage caused by hypoxic or ischemic stress in the ex-vivo Langendorff model through preconditioning mechanisms involving protein kinase C heat shock protein-27 and inducible nitric oxide synthase. Some other drugs including antioxidants have previously been tested for preventing RPE cell damage from oxidative stress. Such drugs have not been sufficiently potent in their anti-oxidant properties or protective capacities to be effective. Our previous data on sulindac treatment of RPE cells in culture indicated that sulindac protects these cells from oxidant induced damage including that caused by hydrogen peroxide and TBHP as well from oxidative stress caused by hypoxia and re-oxygenation. Because sulindac is highly protective against oxidative stress through activation of endogenous protective processes (preconditioning pathways) it could be a unique therapy for protecting RPE cells in macular degeneration. By understanding how sulindac functions to protect RPE cells against oxidative stress, it should be possible to develop derivatives of sulindac that will be more effective without NSAID activity.

Existing drugs for preventing oxidative stress in RPE cells are limited because they are not sufficiently potent as antioxidants. Sulindac has the advantage that by activating endogenous protective mechanisms in normal cells it elicits a very substantial protection against oxidative stress from either oxidants or from hypoxia/ischemia. We have demonstrated that sulindac protects RPE cells in culture against oxidative stress from the oxidants hydrogen peroxide and TBHP as well as from hypoxia with reoxygenation. An additional advantage of sulindac is the fact that the mechanism of protection by sulindac of normal cardiac cells against oxidative stress has been established. This contributes to an understanding as to how sulindac is working in the present experiments and may lead to new drugs for protecting RPE cells against oxidative damage.

Sulindac may be the first new drug in many years that effectively protects RPE cells against oxidative stress in macular degeneration. Because sulindac is highly potent as a protective agent against oxidative stress through activation of endogenous protective mechanisms it is likely to become the drug of choice for treating macular degeneration. In addition it should be possible to develop even better drugs based on the drug's mechanism of action. The applications of the methods and compositions described herein include protecting retinal cells (e.g., RPE cells) against oxidative stress. This applies, for example, to macular degeneration where protection of RPE cells will be therapeutic in preventing the disease. Drugs in the market place are generally directed against new pathological blood vessel formation in macular degeneration which otherwise leads to vessel rupture, retinal bleeding and blindness. Sulindac has the advantage of protecting RPE cells against killing from oxidative stress and by increasing cell survival it is likely to prevent the initiation and progression of the disease through the properties of sulindac that are unrelated to its NSAID activity. An advantage of sulindac is that there is some basic information on the mechanism by which it protects cells against oxidative damage. This could lead to more effective drugs. For example, one compound was developed, as described in more detail below, that is related to sulindac and is not an NSAID that may be used in the methods and compositions described herein.

The ability of sulindac to protect RPE cells against oxidative stress was determined by treating cultured RPE cells with sulindac, before exposing them to oxidative stress. Following 48 h exposure of RPE cells to sulindac, cells were exposed to either a range of tert-Butyl Hydrogen peroxide (t-BHP) concentrations or Hydrogen peroxide (H₂O₂) to induce oxidative stress. For inducing Hypoxia, RPE cells were exposed to less than 0.5% oxygen environment in a hypoxia chamber. After the treatments cellular viability was determined using the MTT assay.

The results show that exposure of cultured RPE cells to oxidative stress using t-BHP or H₂O₂ or Hypoxia causes significant decrease in cell viability. Pretreatment of RPE cells with sulindac for 48 hrs, protects them against these insults and enhances survival. In conclusion, sulindac represents a novel therapeutic agent for oxidative stress induced ocular diseases.

The pharmaceutical compositions described herein can include sulindac, sulindac metabolites, or sulindac derivatives. The pharmaceutical compositions may be implemented in connection with a treatment kit. In one example of such a kit, the kit includes a composition having the sulindac formulated as an eye drop formulation at an effective concentration. The kit may also include educational materials for optimum patient compliance and follow-up. The compositions may be administered to a subject by any suitable delivery route. In a typical embodiment, the compositions are administered as an eye drop formulation. In another embodiment, the composition is administered orally. For oral administration, the daily dose of sulindac may be in the range of about 0.2 mg per kg of body weight to about 1.2 mg per kg of body weight (e.g., a 70 kg subject would be administered about 50-60 mg of sulindac per day). The compositions described herein are preferably administered to a subject in an effective amount. An effective amount is an amount which is capable of producing a desirable result in a treated animal or cell (for example, to protect RPEs from oxidative stress). As is well known in the medical and veterinary arts, dosage for any one animal depends on many factors, including the particular animal's size, body surface area, age, the particular composition to be administered, time and route of administration, general health, and other drugs being administered concurrently.

### EXAMPLES

The present invention is further illustrated by the following specific examples. The examples are provided for illustration only and should not be construed as limiting the scope of the invention in any way.

### Example 1

### Use of Sulindac for Protecting RPE Cells Against Oxidative Stress

### Methods

For protecting RPE cells from oxidative stress induced damage we used the NSAID, sulindac. For our experiment we used ARPE-19 cells cultured in 96 well plates. The cells were preincubated with sulindac for 48 hours prior to exposing them to oxidative stress. Our oxidizing agents of choice were TBHP and Hydrogen peroxide (H₂O₂). We also stressed the ARPE-19 cells with 24 hours of Hypoxia treatment. After the stress the cellular viability was evaluated using the MTS assay. This assay uses a tetrazolium compound that is bioreduced into a formazan product in the cells. The signal generated (color intensity) is directly proportional to the number of viable (metabolically active) cells in the wells.

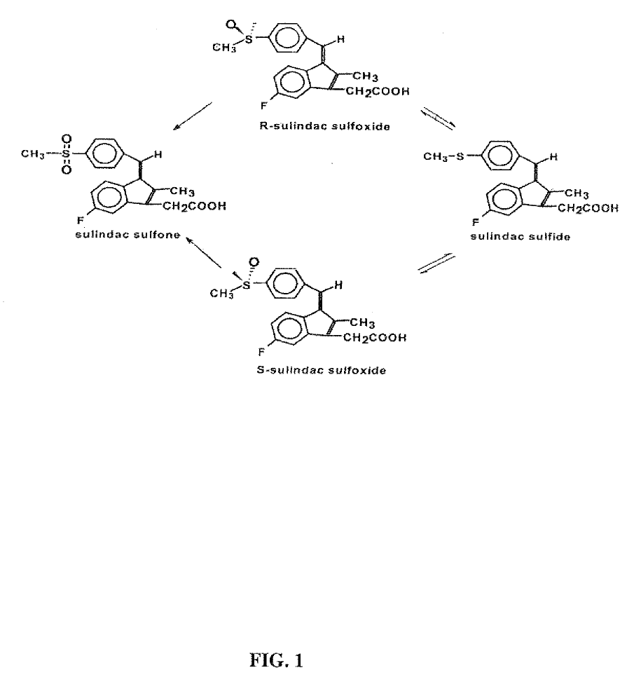

FIG. 1 shows the structure of sulindac and its analogues. Sulindac can be reduced to an active NSAID sulfide form or irreversibly oxidized to a sulfone analogue. The sulfoxide form of sulindac retains the functions of NSAID through COX inhibitory properties.

### Results

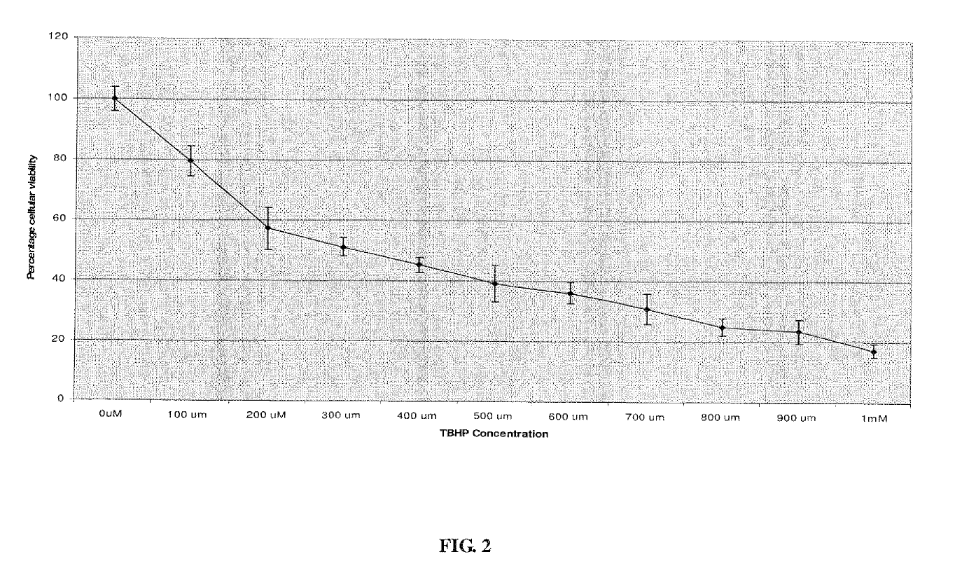

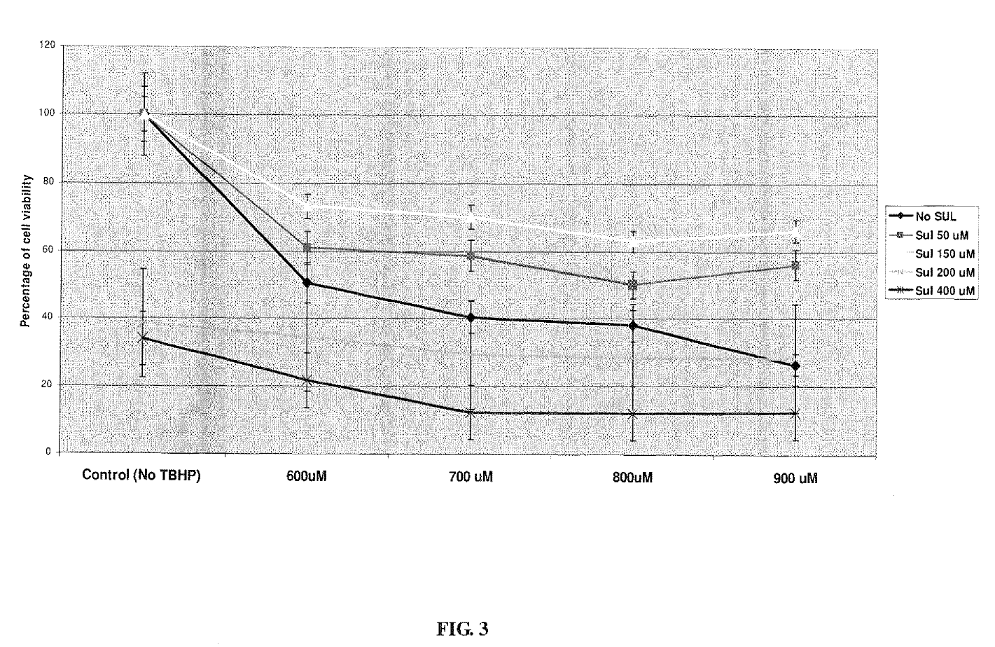

When ARPE-19 cells grown in 96 well plates were exposed to TBHP without prior incubation to sulindac, they exhibited loss of cell viability. FIG. 2 shows a “Kill curve.” The loss of cell viability in retinal pigment epithelial cells exposed to oxidative stress by TBHP treatment was examined. ARPE-19 cells were preincubated with different concentrations of sulindac ranging from 50 uM to 400 μM. The results showed that preincubation with sulindac protected the ARPE-19 cells against loss of cell viability due to oxidative stress. However, sulindac concentrations of 200 μM or higher was found to be toxic to ARPE-19 cells as shown by acute reduction of cell viability even without any TBHP (FIG. 3). FIG. 3 shows results from experiments in which treating ARPE 19 cells with sulindac prior to TBHP exposure prevented cell death caused by oxidative stress.

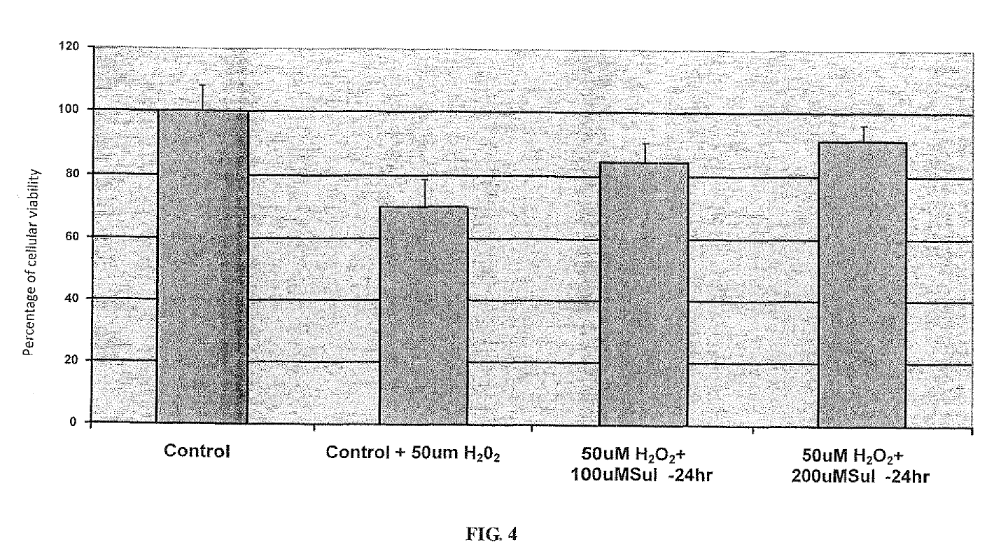

The next experiment was designed to determine if preincubation with sulindac is also effective in protecting ARPE-19 cells against oxidative stress induced by treatment of retina cells with H₂O₂. Cells were incubated with sulindac for 24 hrs and then exposed to 50 μM H₂O₂. The experiment showed that sulindac is capable of protecting retina cells against H₂O₂ induced oxidative stress. The protective effect of sulindac sulfone was also tested under similar conditions and it offered only partial protection (FIG. 4). FIG. 4 shows that the loss of cell viability by H₂O₂ induced oxidative stress can be reduced by preincubating retina cells with sulindac.

To test the ability of sulindac in protecting against a variety of stresses, ARPE-19 cells were exposed to 24 hours of hypoxic stress following sulindac preincubation. The ARPE-19 cells were grown in 96 well plates inside a hypoxia chamber having only 5% oxygen for 24 hours to induce hypoxic stress. Prior to exposing the cells to hypoxia, one set of cells was incubated 24 hours with sulindac. When compared with control cells, the results show that sulindac pretreatment confers significant protection against hypoxia induced stress.

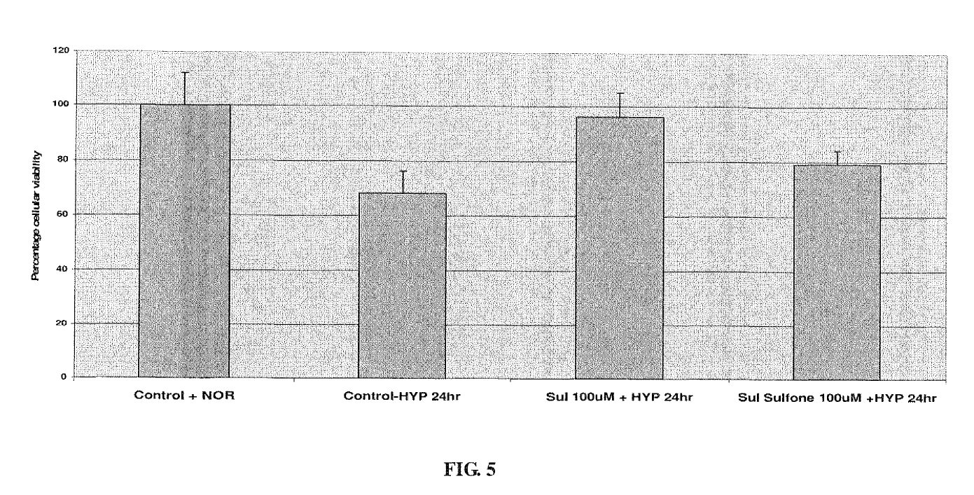

FIG. 5 shows comparative cellular viability of ARPE-19 cells treated with sulindac, sulindac sulfone and untreated (control) cells. The cells preincubated with sulindac showed higher viability when exposed to 24 hr hypoxic stress. The difference in survival between control and sulindac treated cells was statistically significant (P<0.05).

### Summary

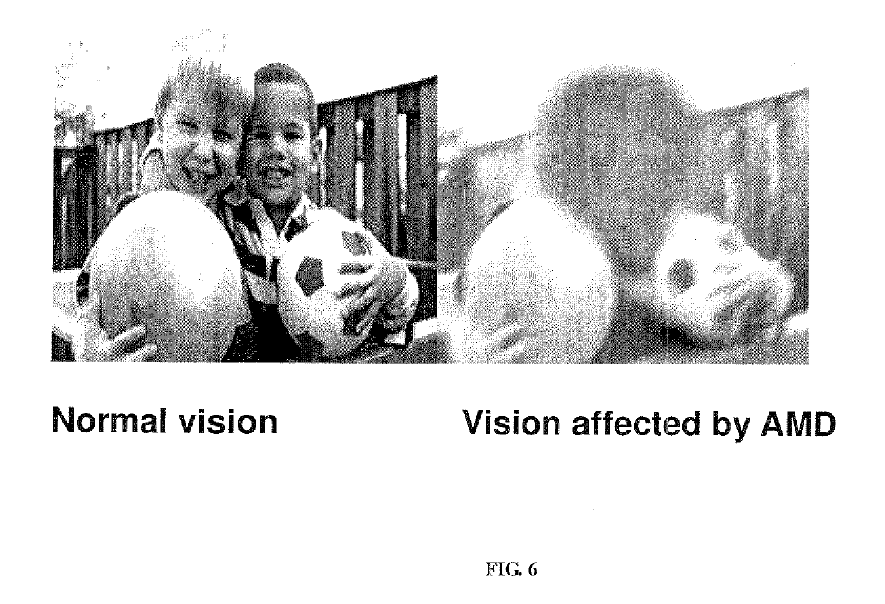

Sulindac increases cell viability in RPE cells exposed to the oxidizing agents TBHP and H₂O₂. Sulindac protects RPE cells against hypoxia-induced cell death. Sulindac sulfone, an analogue of sulindac, shows partial protection in hypoxic conditions. Protection provided to RPE cells by sulindac possibly involves a preconditioning mechanism that influences the mitochondria. Oxidative stress has been implicated as the underlying cause in the damage to various ocular diseases such as cataract, age related macular degeneration (AMD), glaucoma and diabetic retinopathy. AMD is the leading cause of legal blindness in 50 years of age or older. It damages the macula leading to loss of central vision. In FIG. 6, the two images shown highlight the difference in vision caused by damage to the macula in the eyes of patients suffering from AMD.

### Example 2

### Sulindac Protects RPE from Oxidative Stress

The retinal pigment epithelial (RPE) layer is affected by oxidative stress in several retinal diseases. We tested the non-steroidal anti-inflammatory compound (NSAID) sulindac for protection against oxidative stress induced damage in RPE cells. Besides its known anti-inflammatory activity, recent studies have shown that sulindac can protect cardiac cells against oxidative damage by a preconditioning mechanism, independent of its NSAID activity.

The ability of sulindac to protect RPE cells against oxidative stress was determined by analyzing cellular viability following treatment of cultured ARPE19 cells with sulindac, before exposing the cells to conditions leading to oxidative damage. Following 24 hr treatment of the cells with sulindac, ARPE19 cells were exposed to either a range of tert-Butyl Hydrogen peroxide (t-BHP) concentrations for 24 hrs or a range of intensities of ultraviolet B (UVB) to induce oxidative stress. To test the involvement of PKC, the non-specific PKC blocker chelerythrine and also specific blockers for individual isoforms of PKC were used. The possible role of PKG was investigated by the addition of PKG inhibitor Rp-8-Br-PET-CGMPs along with sulindac. Western blotting was used to detect induction of late phase markers of preconditioning.

Exposure of cultured ARPE19 cells to t-BHP or UVB resulted in a substantial decrease in cell viability. Pretreatment of the RPE cells with sulindac for 24 hrs protected them against both types of insult and enhanced survival. The protective effect offered by sulindac was significantly reversed when blocked by chemical inhibitors of either PKG or PKC. Results of the western blotting experiments indicated an upregulation in the levels of Hsp27 and iNOS, two markers of late phase preconditioning.

In summary, the protection provided to ARPE19 cells by sulindac against oxidative stress involves a preconditioning pathway and is unrelated to the NSAID property of sulindac. Experiments are in progress Lo test the protective efficacy of sulindac in an in vivo mouse model of retinal degeneration due to extended light exposure. This study suggests sulindac represents a novel therapeutic agent to treat retinal diseases that arise from oxidative stress.

### Example 3

### Sulindac Protects RPE Cells Against Oxidative Damage

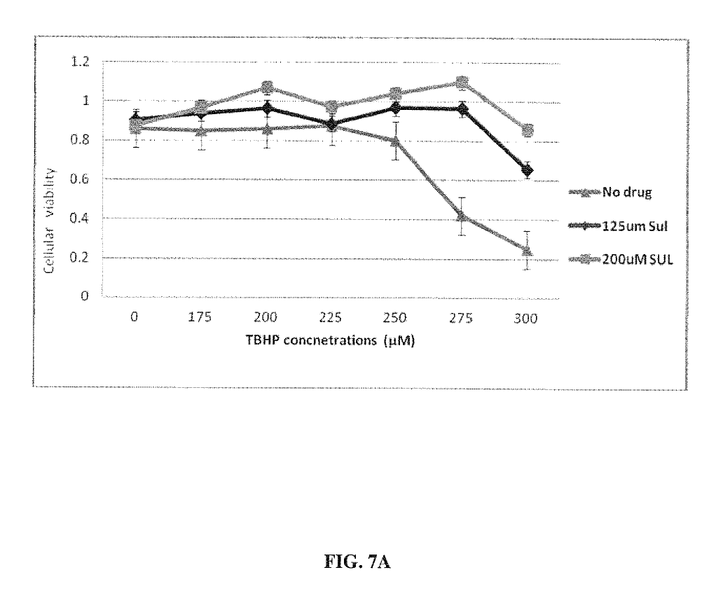

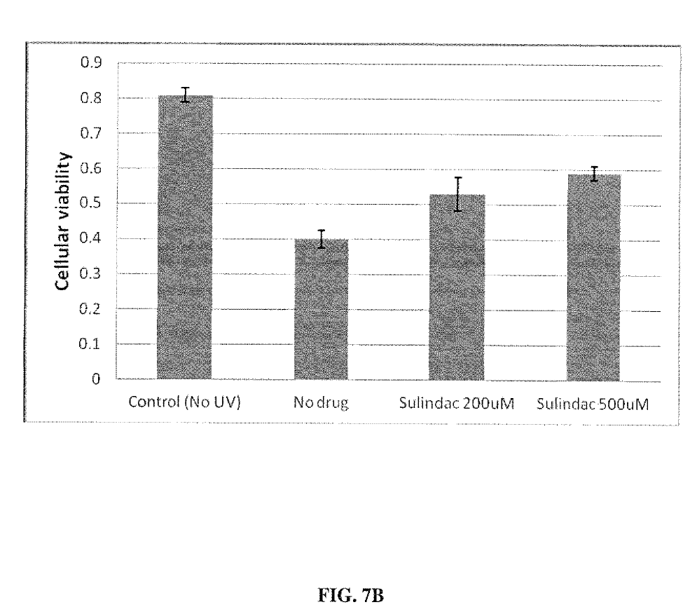

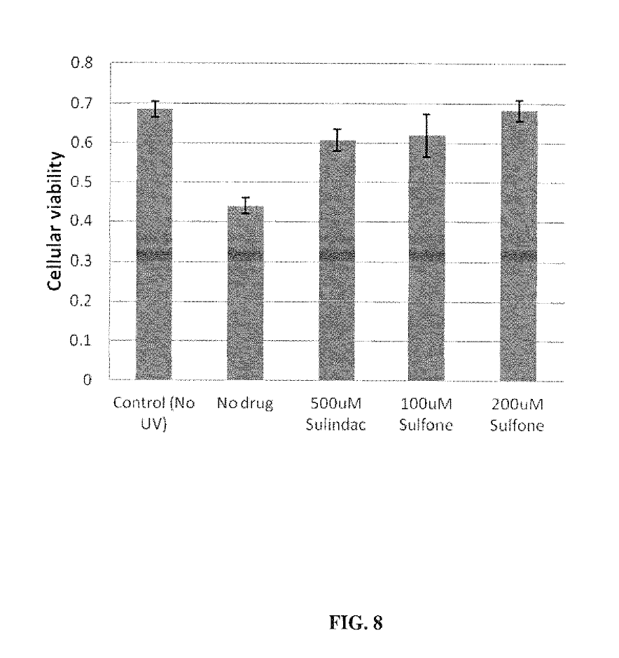

Previous studies showed that sulindac is an ischemic preconditioning agent that can protect cardiac tissue against ischemia/reperfusion damage. It seemed reasonable to investigate whether sulindac could also protect other cells, such as RPE cells, which are susceptible to oxidative damage and known to have a strong ischemic preconditioning (IPC) response. Two types of oxidative stress were used in these experiments, either exposure of the RPE cells to an oxidizing agent such as TBHP, or exposure to UV light. In these experiments the RPE cells were pretreated for 24 hours with varying concentrations of sulindac, sulindac sulfone or sildenafil as described in the Figures. FIG. 7A shows the effect of sulindac in protecting RPE cells against varying concentrations of TBHP as measured by cell viability, whereas FIG. 7B shows the protection of the RPE cells against UV damage by sulindac, using 1200 mjoules of UV radiation. As seen in FIG. 7A sulindac at concentrations of 125 and 200 uM afforded essentially complete protections against TBHP damage, FIG. 7B shows that sulindac, at 500 uM can provide 50% protection against 1200 mj of UV exposure. To determine whether this protective effect was due to the NSAID activity of sulindac, sulindac sulfone, a metabolite of sulindac that has no NSAID activity was tested in place of sulindac for its UV protective effect. These results are shown in FIG. 8. Sulindac sulfone at 200 uM concentration showed complete protection against UV damage. It should also be noted that sulindac sulfone is not a substrate for the Msr system which eliminates the possibility that the sulindac protective effect was related to its being a substrate for the Msr enzymes and functioning as a catalytic anti-oxidant in an ROS scavenging system.

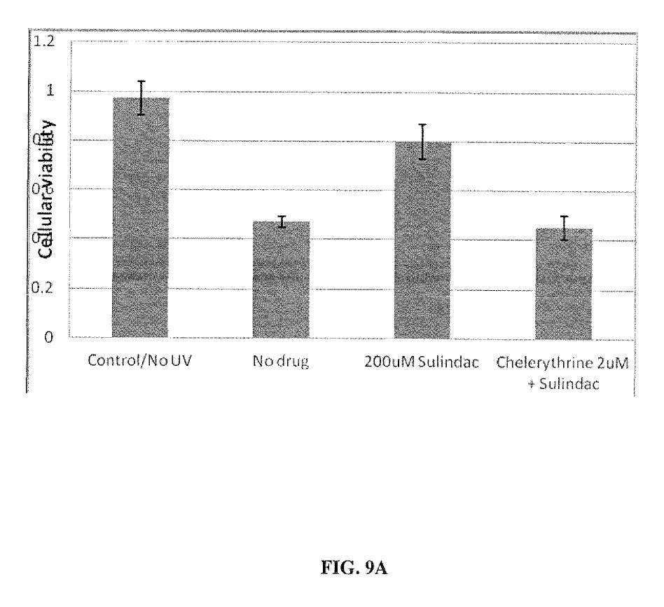

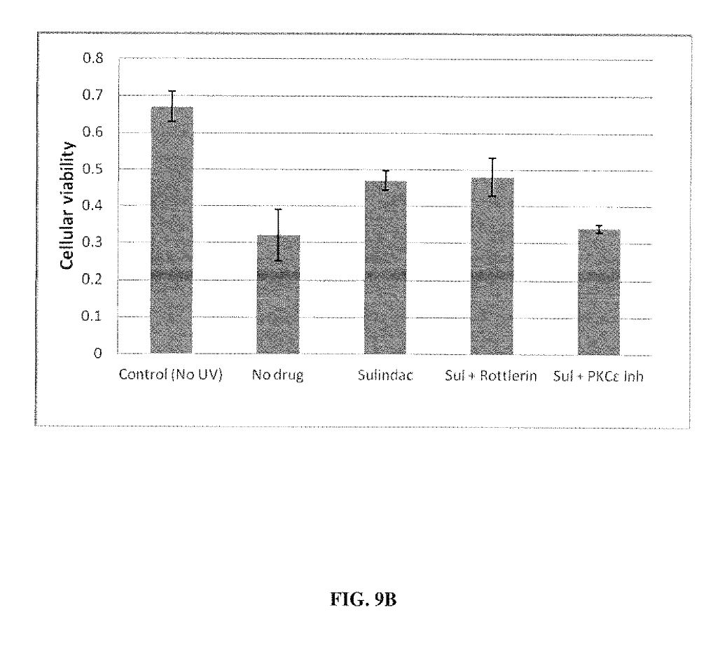

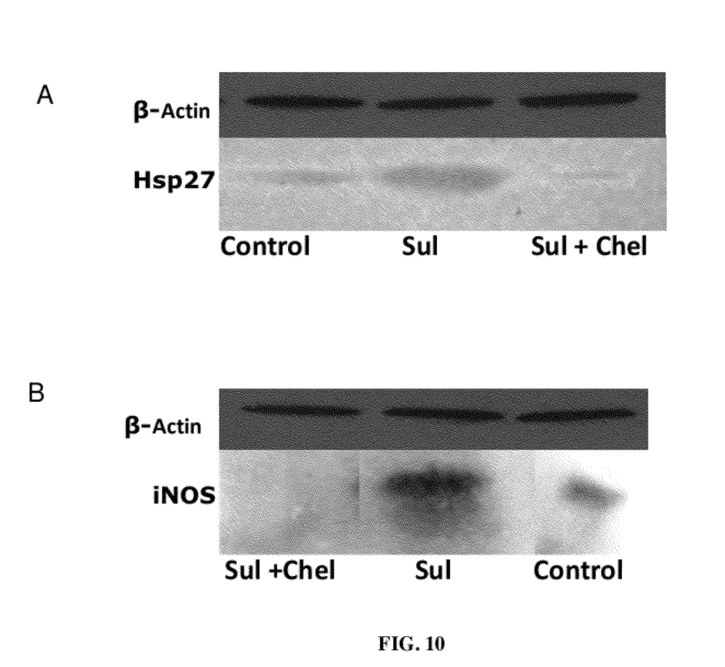

The sulindac protective effect on RPE cells is due to ischemic preconditioning. The IPC response in tissues is often initiated by ROS and/or NO which activates mitochondrial PKC epsilon as part of a complex mechanism that also involves the opening of the mitochondrial ATP sensitive K+ channel and preventing the formation of the mitochondrial permeability transition pore. We have looked more closely at the possible role of PKC in the protection of RPE cells by sulindac, as was shown previously in cardiac studies. As shown in FIG. 9A, chelerythrin, a broad spectrum PKC inhibitor, significantly reversed the protective effect of sulindac against UV damage, suggesting that one or more isoforms of PKC were involved in the sulindac protective effect. Based on previous preconditioning studies it seemed reasonable to look specifically at PKC epsilon. As shown in FIG. 9B, V1-V2, a peptide inhibitor of PKC epsilon almost completely reversed the protective effect of sulindac. In contrast, rottlerin, a PKC delta inhibitor, when used at 3 uM, a concentration reported to inhibit PKC delta, showed no reversal of the sulindac protection. In the previous cardiac study it was also shown that two late stage preconditioning markers, iNOS and Hsp27 were induced by sulindac. As shown in FIG. 10A and FIG. 10B there was significant induction of iNOS and Hsp27 in RPE cells pretreated for 48 hours with sulindac, that was dependent on PKC. These results indicate that sulindac is protecting the RPE cells by initiating an ischemic preconditioning response.

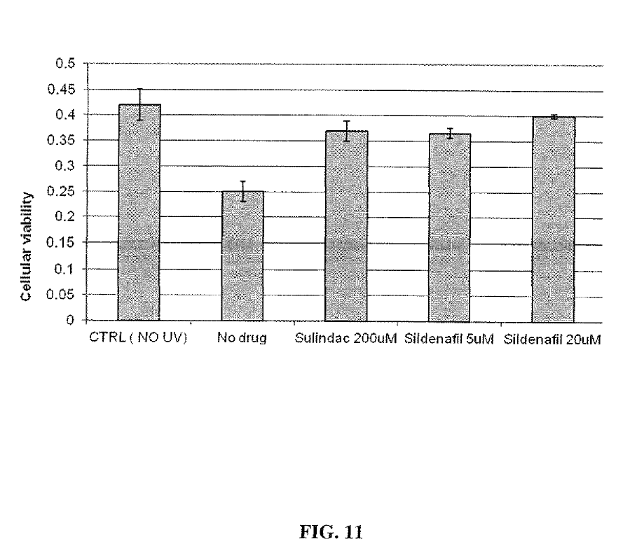

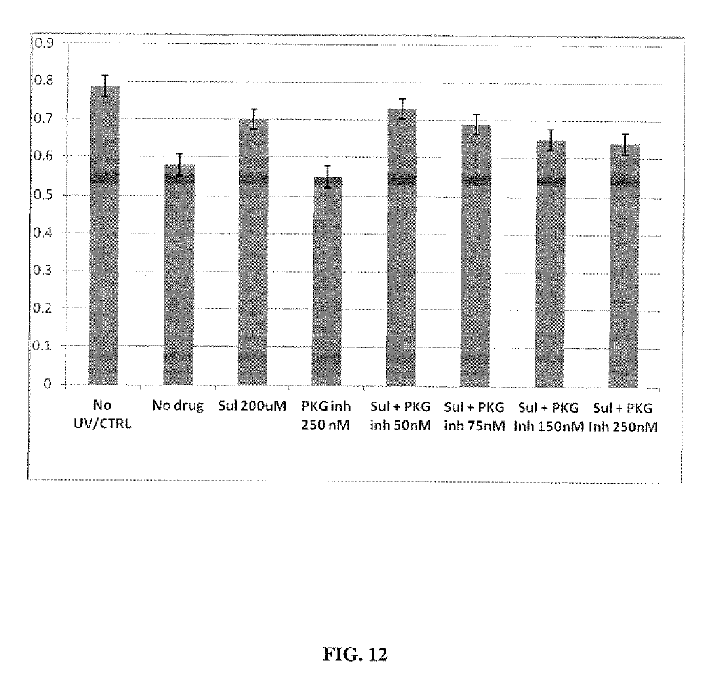

The role of PKG in the sulindac protective effect was examined. The above results support previous studies that agents that can initiate an ischemic preconditioning response may have therapeutic value in delaying the onset, or slowing the progression, of retinal diseases arising from ischemia and oxidative damage. Sildenafil (viagra) is a known ischemic preconditioning agent that has been shown to be a cardio-protectant, but has not been tested as a protective agent for RPE cells. As seen in FIG. 11 sildenafil (20 uM) can also protect RPE cells against UV damage similar to sulindac. Both sulindac and sildenafil are known to be inhibitors of PDE5, which results in increasing the level of cGMP and activation of PKG. Activation of PKG has been shown to initiate IPC in cardiac cells and if sulindac is acting through the PKG activation, inhibitors of PKG should reverse the sulindac protective effect. As shown in FIG. 12, incubation of the cells for 24 hours with sulindac and Rp-8-Br-PET-CGMPs(250 nM), a known inhibitor of PKG, resulted in >50% inhibition of the sulindac protective effect on RPE cells exposed to UV damage. Attempts to completely reverse the sulindac effect by increasing the level of inhibitor were not successful, and one must consider the possibility that the sulindac effect may not only involve PKG, but other, as yet, unidentified agents and pathways.

### Other Embodiments

Any improvement may be made in part or all of the compositions and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of protecting retinal cells from damage from oxidative stress in a subject comprising administering to the subject a composition comprising sulindac in an amount effective to protect retinal cells from damage from oxidative stress.

2. The method of claim 1, wherein the subject is a human with macular degeneration.

3. The method of claim 1, wherein the composition is an eye drop formulation.

4. The method of claim 1, wherein the retinal cells are retinal pigment epithelial (RPE) cells.

5. A pharmaceutical composition comprising sulindac in an amount effective to protect RPE cells from damage from oxidative stress and a pharmaceutically acceptable carrier, the composition formulated as an eye drop formulation.

6. A kit for treating macular degeneration in a subject, the kit comprising:
a) an eye drop formulation comprising sulindac in an amount effective to protect RPE cells from damage from oxidative stress and a pharmaceutically acceptable carrier;
b) instructions for use; and
c) packaging.
